(19) United States
(12) Patent Application Publication
(10) Pub. No.: US 2015/0190652 A1
(43) Pub. Date: Jul. 9, 2015

(54) FLEXIBLE LIPOLYSIS STIMULATING DEVICE

(71) Applicant: Innovate Photonics Limited, Swansea (GB)

(72) Inventor: David Simon George, Swansea (GB)

(73) Assignee: Innovate Photonics Limited, Swansea (GB)

(21) Appl. No.: 14/286,256

(22) Filed: May 23, 2014

(30) Foreign Application Priority Data
Jan. 6, 2014 (GB) 1400149.9

(51) Int. Cl. A61N 5/06

(52) U.S. Cl. CPC A61N 5/0625; A61N 2005/0643; A61N 2005/0652

(57) ABSTRACT

A flexible lipolysis stimulating device and arrangement including a flexible substrate conformable with contours of a user's body, a plurality of radiation generating devices disposed upon the flexible substrate and a flexible housing conformable with the contours of the user's body, which is arranged to house the flexible substrate and the plurality of radiation generating devices.

### CROSS-REFERENCE TO RELATED APPLICATIONS

This patent application claims the benefit under 35 U.S.C. §119 of United Kingdom Patent Application No. 1400149.9, filed Jan. 6, 2014, which application is incorporated herein by reference.

### FIELD OF THE INVENTION

The present invention relates to a flexible lipolysis stimulating device.

### BACKGROUND OF THE INVENTION

When energy is required by the body, the brain sends signals to the adipose cells to break down the stored fat—a process called lipolysis. During this process, free fatty acids are released into the bloodstream and circulate throughout the body.

Physical exercise requires energy and often involves the lipolysis process, and thus facilitates the loss of excess fat and weight. However, the breakdown of fat for the release of energy generally only occurs once the more readily available energy in the form of glucose has been exhausted and as such, prolonged periods of exercise are typically required before the bodily fat resources are utilised.

We have now devised a flexible lipolysis stimulating device for encouraging the breakdown of fatty stores for subsequent use by the body.

### BRIEF SUMMARY OF THE INVENTION

In accordance with a first aspect of the present invention, there is provided a flexible lipolysis stimulating device comprising a flexible substrate conformable with the contours of the user's body, a plurality of radiation generating devices disposed upon the substrate and a flexible housing conformable with the contours of the users body, which is arranged to house the substrate and radiation generating devices.

The stimulating device further comprises a power source communicatively coupled with the radiation generating devices for powering the radiation generating devices to emit radiation. In an embodiment, the power source may comprise a battery source disposed upon the substrate. In an alternative embodiment, the power source may comprise a mains power supply.

The radiation generating devices may comprise light emitting diodes (LEDs) which are arranged to generate radiation in the wavelength range of 600-900 nm. In an embodiment, a first portion of the LEDs may be arranged to generate radiation having a wavelength of 635 nm for example, which may penetrate to a depth of 10 mm into the dermis and a second portion of the LEDs may be arranged to generate radiation having a wavelength of 900 nm, which may penetrate the dermis to a depth of approximately 25 mm. The radiation stimulates mitochondria in the cells of the dermis at different depths thereof, and this causes a temporary charge in the cells which in turn releases a lipase enzyme into the cell breaking down the fat into fatty acids, glycerol and water. These then pass through the cell wall and are picked up by the lymphatic system for use as energy.

In a further embodiment, the LEDs may alternatively be arranged to generate both 635 nm and 900 nm radiation.

In an embodiment, the housing comprises a front section and a rear section which are coupled together to encapsulate the substrate and radiation generation devices. The front cover preferably comprises a plurality of apertures formed therein which are formed at positions corresponding to the location of the radiation generation devices upon the substrate. This enables the radiation generated by the radiation generation devices to pass out from the housing and treat the desired area of the user's body.

The stimulating device preferably further comprises a cover which is detachably couplable with the housing and which is arranged to extend over the front section thereof. The cover is preferably formed of a material which is substantially transmissive for the wavelength of radiation generated by the radiation generating devices. The cover preferably further comprises a plurality of lensing regions formed thereon which are located at positions corresponding to the apertures formed in the front section of the cover. The lensing regions are arranged to receive the divergent rays of radiation emanating from the radiation generating devices and re-direct the rays so that they propagate from the lensing regions in substantially parallel directions.

In an embodiment, the lensing regions comprise Fresnel lenses.

In an embodiment, the cover comprises an adhesive portion on a front side thereof for enabling detachable coupling of the cover to the front section of the housing and an adhesive portion disposed on a rear side thereof for enabling detachable coupling of the stimulating device to a portion of the users body.

In accordance with a second aspect of the present invention, there is provided a lipolysis stimulating arrangement comprising a plurality of flexible lipolysis stimulating devices of the first aspect and a docking station for enabling the stimulating devices of the first aspect to removably dock therewith.

These and other objects and advantages of the present invention will be readily appreciable from the following description of preferred embodiments of the invention and from the accompanying drawings and claims.

### BRIEF DESCRIPTION OF THE DRAWINGS

The nature and mode of operation of the present invention will now be more fully described in the following detailed description of the invention taken with the accompanying drawing figures, in which:

FIG. 1 is an exploded view of a flexible lipolysis stimulating device according to a first embodiment of the present invention;

FIG. 2 is an exploded view of a flexible lipolysis stimulating device according to a second embodiment of the present invention; and,

FIG. 3 is a schematic illustration of a lipolysis stimulating device according to an embodiment of the present invention.

### DETAILED DESCRIPTION OF THE INVENTION

At the outset, it should be appreciated that like drawing numbers on different drawing views identify identical, or functionally similar, structural elements of the invention. While the present invention is described with respect to what is presently considered to be the preferred aspects, it is to be understood that the invention as claimed is not limited to the disclosed aspects.

Furthermore, it is understood that this invention is not limited to the particular methodology, materials and modifications described and as such may, of course, vary. It is also understood that the terminology used herein is for the purpose of describing particular aspects only, and is not intended to limit the scope of the present invention, which is limited only by the appended claims.

Unless defined otherwise, all technical and scientific terms used herein have the same meaning as commonly understood to one of ordinary skill in the art to which this invention belongs. Although any methods, devices or materials similar or equivalent to those described herein can be used in the practice or testing of the invention, the preferred methods, devices, and materials are now described.

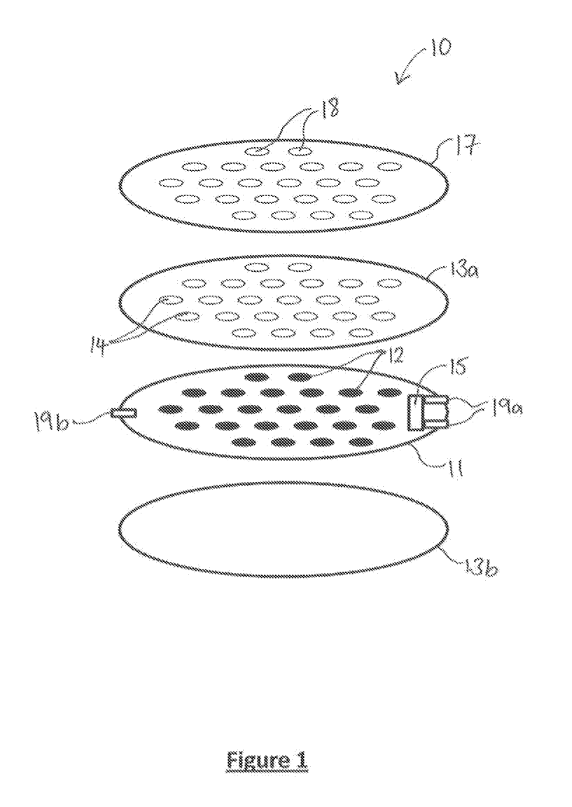

Referring to FIG. 1 of the drawings, there is illustrated a flexible lipolysis stimulating device 10 according of a first embodiment of the present invention for facilitating the breakdown of body fat. The device 10 comprises a flexible substrate 11 which may be formed of a plastics material, and upon which are mounted a plurality of radiation generating devices 12. The radiation generating devices 12 are equally spaced upon the substrate 11 and are arranged to deliver radiation to the portions of the users body (not shown) which require the breakdown of fat. The radiation generating devices 12 may comprise a plurality of light emitting diodes (LEDs) and are arranged to generate radiation in the wavelength range of 600-900 nm and thus generate both visible light and heat. The shorter 600 nm wavelength radiation is found to penetrate the dermis of the body to a depth of approximately 10 mm, whereas the 900 nm wavelength radiation extends to a greater depth of approximately 25 mm. The radiation stimulates mitochondria in the cells of the dermis at different depths thereof and causes a temporary charge in the cells which in turn releases a lipase enzyme into the cell breaking down the fat into fatty acids, glycerol and water. These then pass through the cell wall and are picked up by the lymphatic system for use as energy during exercise. In an embodiment, the plurality of LEDs 12 may comprise a portion which are arranged to generate radiation at a particular wavelength, however, in an alternative embodiment, each LED may be arranged to generate radiation across the wavelength range of 600-900 nm.

The flexible lipolysis stimulating device 10 further comprises a flexible housing 13 which may be formed of a plastics and/or foam material. The housing 13 comprises a front section 13a and a rear section 13b which are detachably coupled together to substantially encapsulate the substrate 11 and radiation generating devices 12. The front section of the housing 13a comprises a plurality of apertures 14 formed therein which are located at positions corresponding to the location of the LEDs 12 upon the substrate 11. In this respect, the radiation generated by the LEDs 12 is permitted to pass out from the housing 13 to treat the desired portion of the user's body (not shown).

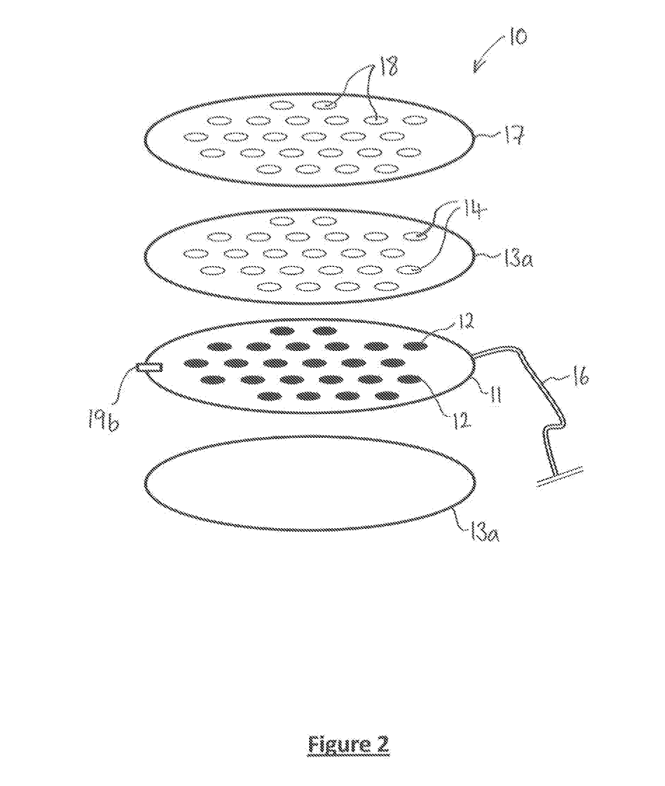

The stimulating device 10 further comprises a power source for powering the LEDs 12. In the first embodiment (as illustrated in FIG. 1 of the drawings), the power source may comprise a battery source 15, which may be rechargeable, disposed upon the substrate 11. In a second embodiment of the present invention as illustrated in FIG. 2 of the drawings, the power source may comprise the mains supply (not shown), which is delivered to the LEDs 12 via an electrical cable 16, for example.

The stimulating device 10 further comprises a cover 17, detachably coupled over the front section 13a of the housing 13. The cover 17 is formed of a flexible material which is substantially transmissive to radiation in the wavelength range of 600-900 nm and thus allows the radiation generated by the LEDs to pass therethrough substantially unattenuated. The cover 17 comprises a plurality of lensing regions 18 formed thereon which are located to correspond with the location of the apertures 14 on the front housing section 13a. The lensing regions 18 comprise Fresnel lenses which may be formed integrally with the cover 17 and serve, in use, to substantially align the divergent rays of radiation incident thereof from the adjacent LED 12, such that the rays of radiation which propagate from the stimulating device 10 are substantially parallel.

The cover 17 further comprises adhesive portions (not shown) disposed on a front side thereof for enabling the cover 17 to detachably couple with the front section 13a of the housing 13, and also adhesive portions (not shown) on the rear side thereof for enabling the device 10 to be detachably coupled to the region of the users body (not shown).

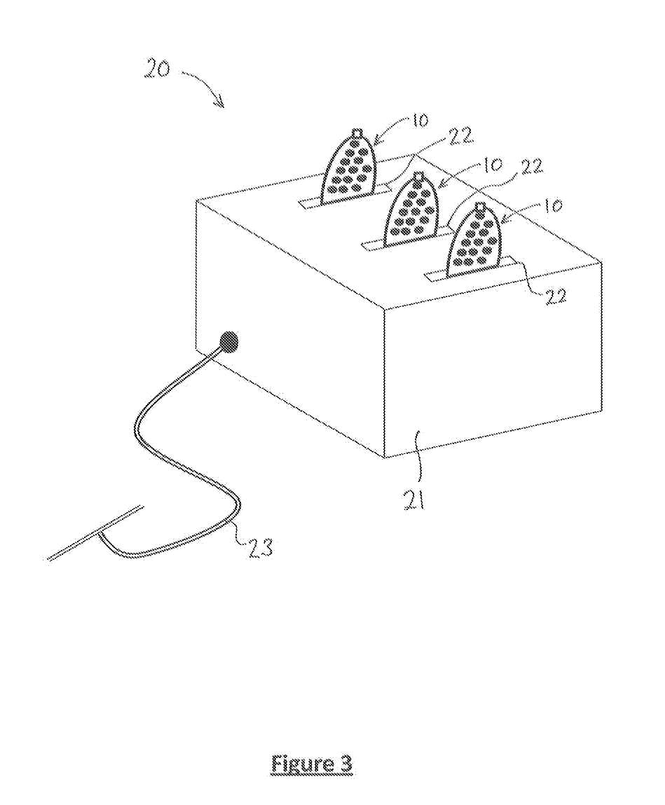

Referring to FIG. 3 of the drawings, there is illustrated a lipolysis stimulating arrangement 20 according to an embodiment of the present invention. The arrangement 20 comprises a plurality of flexible lipolysis stimulating devices 10 described above which are permitted to removably dock within docking regions 22 of a docking station 21. The docking station 21 provides for a secure housing of the stimulating devices 10 when not in use, and in an embodiment, may also comprise electrical contacts (not shown) within each docking region 22 for coupling with electrical contacts 19a disposed on the stimulating devices of the first embodiment, for permitting a recharging of the battery source 15 when the devices 10 are docked therein. In a further embodiment, the docking station 21 may comprise a reel (not shown) associated with each docking region 22 for permitting a retractable extension of the electrical cables 16 between the docking station 21 and the stimulating devices 10.

The docking station 21 may further comprise an electrical cable 23 for coupling the docking station 21 to a mains power supply (not shown), so that electrical power can be transferred to the stimulating devices 10, via the docking station 21.

In use, the stimulating devices 10 are secured to the region of the users body requiring treatment and the flexible nature of the substrate 11, housing 13 and cover 17 permit the devices 10 to conform with the contours of the body region to provide for an improved coupling of the radiation into the dermis, compared with substantially rigid stimulating devices. The devices 10 may be secured in place using a strap (not shown). Alternatively, the devices 10 may be secured in place via the adhesive portions (not shown) disposed on the rear side of the cover 17. Once the devices 10 have been secured in place, the devices 10 may then be powered by operating a switch 19b disposed on each device 10.

The flexible nature of the stimulating devices 10 provide for an improved breakdown of body fat by providing an enhanced coupling of the radiation from the LEDs 12 into the dermis, and thus facilitate an improved weight loss and control.

Thus, it is seen that the objects of the present invention are efficiently obtained, although modifications and changes to the invention should be readily apparent to those having ordinary skill in the art, which modifications are intended to be within the spirit and scope of the invention as claimed. It also is understood that the foregoing description is illustrative of the present invention and should not be considered as limiting. Therefore, other embodiments of the present invention are possible without departing from the spirit and scope of the present invention.

What I claim is:

1. A flexible lipolysis stimulating device comprising:
a flexible substrate conformable with contours of a user's body;
a plurality of radiation generating devices disposed upon the flexible substrate; and,
a flexible housing conformable with the contours of the user's body, which is arranged to house the flexible substrate and the plurality of radiation generating devices.

2. The flexible lipolysis stimulating device according to claim 1 further comprising:
a power source communicatively coupled with the plurality of radiation generating devices for powering the plurality of radiation generating devices to emit radiation.

3. The flexible lipolysis stimulating device according to claim 2, wherein the power source comprises a battery source disposed upon the flexible substrate.

4. The flexible lipolysis stimulating device according to claim 2. wherein the power source comprises a mains power supply.

5. The flexible lipolysis stimulating device according to claim 1, wherein the plurality of radiation generating devices comprise light emitting diodes.

6. The flexible lipolysis stimulating device according to claim 5, wherein the light emitting diodes are arranged to generate radiation in a wavelength range of 600-900 nm.

7. The flexible lipolysis stimulating device according to claim 1, wherein the flexible housing comprises a front section and a rear section which are coupled together to encapsulate the flexible substrate and the plurality of radiation generation devices.

8. The flexible lipolysis stimulating device according to claim 7, wherein the front section comprises a plurality of apertures formed therein which are formed at positions corresponding to locations of the plurality of radiation generation devices disposed upon the flexible substrate.

9. The flexible lipolysis stimulating device according to claim 8 further comprising:
a cover which is detachably couplable with the flexible housing.

10. The flexible lipolysis stimulating device according to claim 9, wherein the cover comprises a plurality of lensing regions formed thereon which are located at positions corresponding to the plurality of apertures formed in the front section of the cover.

11. The flexible lipolysis stimulating device according to claim 10, wherein the lensing regions comprise Fresnel lenses.

12. The flexible lipolysis stimulating device according to claim 9, wherein the cover comprises an adhesive portion on a front side thereof for enabling detachable coupling to the front section of the cover and an adhesive portion disposed on a rear side thereof for enabling detachable coupling of the stimulating device to a portion of the user's body.

13. The flexible lipolysis stimulating device according to claim 1 further comprising:
a cover which is detachably couplable with the flexible housing.

14. A lipolysis stimulating arrangement comprising:
a plurality of flexible lipolysis stimulating devices, wherein each of the plurality of flexible lipolysis stimulating devices comprises:
a flexible substrate conformable with contours of a user's body;
a plurality of radiation generating devices disposed upon the flexible substrate; and,
a flexible housing conformable with the contours of the user's body, which is arranged to house the flexible substrate and the plurality of radiation generating devices; and,
a docking station for enabling the plurality of flexible lipolysis stimulating devices to removably dock therewith.
